(19) United States
(12) Patent Application Publication
(10) Pub. No.: US 2026/0054007 A1
(43) Pub. Date: Feb. 26, 2026

(54) Injector Pump for Delivery of Hormone Therapy

(71) Applicant: Mei Zhang, Sharon, MA (US)

(72) Inventor: Mei Zhang, Sharon, MA (US)

(21) Appl. No.: 19/308,633

(22) Filed: Aug. 25, 2025

Related U.S. Application Data
(60) Provisional application No. 63/686,351, filed on Aug. 23, 2024.

(51) Int. Cl. A61M 5/142; A61M 5/145; A61M 39/24

(52) U.S. Cl. CPC A61M 5/14248; A61M 5/1452; A61M 39/24; A61M 2005/14208; A61M 2202/0007; A61M 2202/04; A61M 2205/3303; A61M 2205/3327; A61M 2205/3334; A61M 2205/50; A61M 2210/04

(57) ABSTRACT

An injector pump having a fluid reservoir sealed to a length of tubing extending from the reservoir and an automatic needle injection mechanism. The fluid hormone is delivered over a continuous, extended period to provide for the delivery of replacement hormones based on the hormone fluctuations of the patient and naturally occurring hormone cycles.

### CROSS REFERENCE TO RELATED APPLICATION

This application claims the benefit of U.S. Provisional Patent Application No. 63/686,351, filed on Aug. 23, 2024, which is hereby incorporated by reference.

### BACKGROUND OF THE INVENTION

The present invention relates to devices for the automatic delivery of drugs to a patient and, in particular, to an ambulatory drug delivery device allowing for cyclic delivery of hormones over time.

The ability to deliver metered amounts of hormones over time to replace or supplement the human body's natural production of hormones can treat conditions which occur when the body no longer produces the hormone adequately. For example, hormone therapy may be used to replace the reduction of the hormones estrogen and progesterone during menopause. Hormone therapy can be used to treat hot flashes (i.e., sudden, strong feelings of heat and sweating) and vaginal dryness, itching and burning. Certain forms of estrogen can be used to relieve the symptoms of certain types of prostate cancer. Another form of estrogen can also be used to treat abnormal vaginal bleeding.

For this purpose, hormone therapy can be delivered through pills, injections, or skin preparations such as gels, sprays, or patches. Although pills and skin preparations are non-invasive, subcutaneous injections are typically injected into the muscle or vein and thus may produce a higher, more consistent response. However, the injection dose in a single bolus can cause large fluctuations of estrogen levels causing unwanted mood swings, weight gain, hot flashes, anxiety or migraines in the patient.

An improved class of injector pumps may assist with the subcutaneous delivery of drugs by attaching directly to the patient's skin and providing automatic needle insertion and delivery of drugs over time.

### SUMMARY OF THE INVENTION

The present invention provides a wearable injector infusion pump having a disposable drug reservoir comprised of a fluid reservoir sealed to a needle injection system held by a pump housing. The fluid reservoir and needle injection system provide a robust leak resistant volume that is easily prefilled by the pharmacist through a filling port. The fluid reservoir is contained in a lightweight pump housing facilitating adhesive style attachment to the skin of the patient. The fluid reservoir utilizes the pressure on the fluid reservoir to drive fluid out of the fluid reservoir which is further metered by a metering valve to deliver a drug over an extended time.

The pump housing supports sensors or communicates with wearables such as wristwatches with sensors that sense biomarkers indicative of hormone level or hormone level changes such as changes in basal body temperature, blood pressure, galvanic skin response, sweat production, heart rate, respiration variability, and presence of certain biological molecules in the patient's blood, urine and/or sweat.

It is understood that the present invention may be used in connection with the delivery of many types of hormones including estrogen, progesterone, or progestins, and testosterone for hormone replacement therapy, melatonin, insulin, growth hormone, steroid hormone, cortisol, dopamine, oxytocin, adrenaline, aldosterone, androgens, endocrine, somatostatin, prolactin, follicle stimulating hormone, thyroid stimulating hormone, adrenocorticotropic hormone, luteinizing hormone, gonadotropin-releasing hormone, lipid-derived hormones, etc. Many types of hormones and hormone delivery are contemplated by the present invention such as hormone replacement therapy (for menopause), oncologic hormone therapy, androgen replacement therapy, and transgender hormone therapy.

One embodiment of the present invention provides a method of delivering hormones to a human patient, the method comprising attaching an ambulatory pump to the skin of the human patient, the pump having a pump housing attachable to the skin of the human patient, a hypothermic needle supported by the pump housing along an insertion path of the hypothermic needle into the skin of the human patient, a fluid container supported by the pump housing and containing liquid hormones to fluidly communicate with the hypothermic needle to deliver liquid hormones to the hypothermic needle, a flow control valve controlling the flow of liquid hormones through the hypothermic needle, and a controller sending a signal to the flow control valve to control the amount of flow of the liquid hormones through the flow control valve; monitoring real time hormone levels of the human patient; delivering liquid hormones and adjusting the flow of liquid hormones based on comparing real time hormone levels of the human patient and desired set points or targets for hormones levels within the human patient.

It is thus a feature of at least one embodiment of the present invention to monitor hormone changes in a patient and deliver hormones to restore normal hormone levels using an ambulatory pump design.

The method may further comprise detecting changes in the hormone level and distinguishing from predictable fluctuations expected in the human body.

It is thus a feature of at least one embodiment of the present invention to differentiate normal hormone fluctuations from abnormal hormone changes in the patient since many hormone levels are cyclic and tied to biological cycles rather than external factors such as stimuli or stressors.

The method may further comprise detecting decreases in hormones level and increasing the flow of liquid hormones to the human patient.

It is thus a feature of at least one embodiment of the present invention to compensate for hormone losses during aging, such as overall drops in estrogen, testosterone, growth hormone and melatonin levels. The additional hormones can follow biological cycles.

The method may further comprise increasing the flow of liquid hormones to the human patient is at a steady rate over an extended period.

It is thus a feature of at least one embodiment of the present invention to compensate for drops in hormone levels, for example, due to aging, diet and nutrition, or lifestyle in the patient using slow titration.

The method may further comprise increasing the flow of liquid hormones to the human patient in a single bolus to the human patient.

It is thus a feature of at least one embodiment of the present invention to compensate for sudden drops or large deficits in hormone levels, for example, caused by stress, certain medications, tumors, and medical conditions such as autoimmune disease, endocrine gland injury, polycystic ovary syndrome (PCOS), thyroid disorders and diabetes.

The method may further comprise delivering the bolus to the human patient in response to a request from the human patient by pressing a button.

It is thus a feature of at least one embodiment of the present invention to allow the patient to increase hormone levels quickly in emergency situations for quick physiological response.

The method may further comprise using predictive artificial intelligence (AI) technologies to predict changes in the patient's hormone levels before they occur based on small biological changes or imperceptible trends, patterns, and/or correlations more quickly detectable using AI.

It is thus a feature of at least one embodiment of the present invention to reduce the effect of overshooting the desired hormone amount when delivering boluses causing unwanted side effects by anticipating and compensating for hormone drops incrementally before a bolus is necessary.

The ambulatory pump may further comprise an electronic memory storing the desired set points or targets for hormones levels within the human patient.

It is thus a feature of at least one embodiment of the present invention to store target levels within the pump or external to the pump and communicating the data to the pump via a wired or wireless connection.

The desired set points or targets for hormone levels of the patient may be based on a historical average of the patient.

It is thus a feature of at least one embodiment of the present invention to use average historical data of the actual patient to provide accurate set points for the actual patient.

The historical average of the human patient may be based on prior clinical data and testing on the human patient.

It is thus a feature of at least one embodiment of the present invention to use clinical data to determine set points for the patient to customize treatments.

The desired set points or targets for hormone levels of the patient may be based on a historical average of anonymized patient data based on similar age, height and/or weight.

It is thus a feature of at least one embodiment of the present invention to use representative patient data based on similar age, height and/or weight when historical or clinical data for the specific patient is not available.

The method may further comprise monitoring the hormone level of the human patient using a body sensor.

It is thus a feature of at least one embodiment of the present invention to use real time biological data to determine accurate hormone levels.

The body sensor may be a skin sensor measuring body markers of the human patient.

It is thus a feature of at least one embodiment of the present invention to detect hormone levels using non-invasive means such as detecting biomarkers on the patient's skin like body temperature, blood pressure, galvanic skin response, sweat production, heart rate, respiration variability, or biological molecules in the patient's sweat.

The body sensor may be attached to the pump housing.

It is thus a feature of at least one embodiment of the present invention to incorporate hormone detection into the pump housing for ease of use and improved accuracy.

The body sensor may be external to the pump housing.

It is thus a feature of at least one embodiment of the present invention to use body sensors attached to the back of the arm, abdomen, or buttocks for optimal biomarker sensing placement.

An alternative embodiment of the present invention is an ambulatory pump attachable to skin of a human patient comprising a pump housing attachable to the skin of the human patient; a hypothermic needle supported by the pump housing along an insertion path of the hypothermic needle into the skin of the human patient; a fluid container supported by the pump housing and containing liquid hormones to fluidly communicate with the hypothermic needle to deliver liquid hormones to the hypothermic needle; a flow control valve controlling the flow of liquid hormones through the hypothermic needle; and a controller sending a signal to the flow control valve to control the amount of flow of the liquid hormones through the control valve based on desired set points or targets for hormones levels within the human patient.

It is thus a feature of at least one embodiment of the present invention to provide improved delivery of hormones to a human patient which provides real time hormone detection and real time hormone delivery adjustments based on desired set points of the unique patient.

The fluid container may be a flexible fluid pouch. Alternatively, the fluid container may be a syringe.

It is thus a feature of at least one embodiment of the present invention to allow the patient to deliver hormones in a controlled, continuous, or scheduled manner while the patient remains mobile.

The ambulatory pump may further comprise an electronic memory storing the desired set points or targets for hormones levels within the human patient.

It is thus a feature of at least one embodiment of the present invention to allow the delivery of hormones to be programmable and/or mechanical.

The desired set points or targets for hormones levels within the human patient may consider predictable fluctuations expected in the human body.

It is thus a feature of at least one embodiment of the present invention to provide delivery of insulin, growth hormone, or other endocrine treatments in a manner that is lightweight and discreet.

These particular objects and advantages may apply to only some embodiments falling within the claims and thus do not define the scope of the invention.

### BRIEF DESCRIPTION OF THE DRAWINGS

FIG. 1 is a fragmentary, perspective view of the wearable injector pump of the present invention as attached to the abdomen skin of a patient to communicate with a delivery needle, drug port or the like;

FIG. 2 is an exploded, perspective view of the wearable injector pump of FIG. 1 showing a housing for containing a fluid reservoir, integrated IV line, and needle injector system (not shown) and an adhesive film for attachment of the pump to the patient's skin;

FIG. 3 is a schematic of the pump of FIGS. 1 and 2 showing the fluid reservoir communicating through the IV line to a needle of a needle injection system and a valve regulating flow from the fluid reservoir;

FIG. 4. is chart showing the monitoring of actual hormone levels compared to desired hormone levels and the delivery of steady flow rate hormones and a bolus delivery based on real time monitoring of the patient's hormone level;

FIG. 5 is an alternative embodiment of the fluid reservoir of FIGS. 2 and 3 where the fluid reservoir is a flexible fluid pouch interacting with a pretensioned spring; and

FIG. 6 is an alternative embodiment of the fluid reservoir of FIGS. 2 and 3 where the fluid reservoir is a tubular barrel of a syringe interacting with a pretensioned spring.

### DETAILED DESCRIPTION OF THE PREFERRED EMBODIMENT

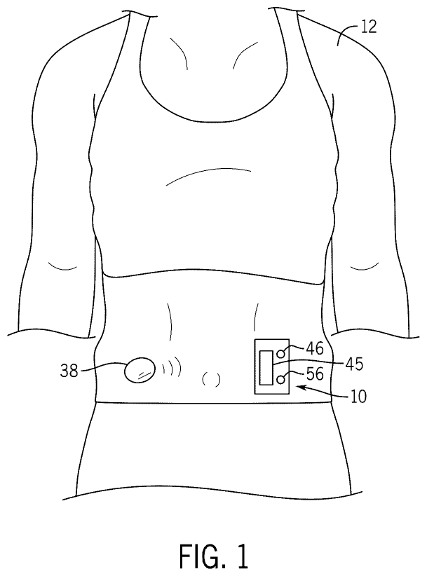

Referring now to FIG. 1, a wearable injector pump 10 according to the present invention may be attached, for example, to abdominal skin of a patient 12 to support the wearable injector pump 10 during normal activities of the patient and to provide the delivery of hormones through a fluid delivery tube 14 connected to a soft needle 16 or another similar element inserted into the patient's skin, for example, to deliver fluid hormones to the muscle. The delivery of fluid hormones can be continuous and regulated over time and adjusted to the fluctuating hormone levels of the patient. The wearable injector pump 10 may be worn for days or weeks (1 to 4 weeks) at a time with minimal discomfort. Other areas of the body where the wearable injector pump 10 may be worn include the arms and legs.

The wearable injector pump 10 may take a small form factor, for example, being approximately one inch by one- and one-half inches in size or smaller so that it can be carried by the patient's skin. In certain embodiments, the infusion pump is not worn on the patient's skin but carried by the patient, for example worn on a strap around the patient's wrist, arm, leg or waist, and communicating through intravenous tubing to deliver the fluid hormones to a delivery needle and/or to a medical port communicating with a large vein as known in the art.

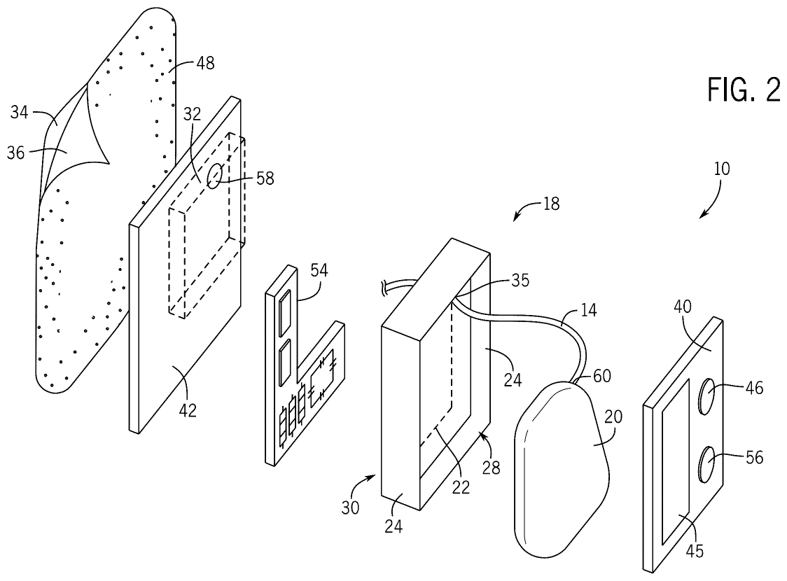

Referring to FIG. 2, the wearable injector pump 10 may include a pump housing 18 supporting therein a fluid reservoir 20 holding the fluid hormone and intended for disposal after single use with a single patient. The pump housing 18 comprises a rigid housing frame having an inner dividing wall 22 surrounded by frontwardly and rearwardly extending sidewalls 26. The frontward extent is positioned away from the skin of a patient 12 and the rearward extent is positioned toward the skin of a patient 12. The dividing wall 22 separates a front volume 28 and rear volume 30 configured to carry the fluid reservoir 20 and the needle injection mechanism 32 and control electronics 54, respectively. The separation of the fluid reservoir 20 from the control electronics 54 components ensures that any accidental leaks from the fluid reservoir 20 do not affect the operation of the control electronics 54.

The frontward extent of the sidewalls 24 defines the front volume 28 into which the fluid reservoir 20 may fit and be stored. At the time of manufacture, the fluid reservoir 20 may be sterilized and evacuated to be later filled by a pharmacist as will be discussed.

The fluid reservoir 20 may have an integrally attached fluid delivery tube 14 extending from the fluid reservoir 20 through an opening 35 in the dividing wall 22 from a front side of the dividing wall 22 to a rear side of the dividing wall 22, permitting fluid to flow from the fluid reservoir 20 held within the front volume 28 to the needle injection mechanism 32 held within the rear volume 30 and near the patient's skin.

Thus, the pump housing 18 supports therein a hormone delivery system which includes but is not limited to the fluid reservoir 20 attached to fluid delivery tube 14 and soft needle 16 that optionally operates alone or in connection with motorized elements, as further described below, and acting as the wearable injector pump 10 to deliver hormone fluids through the fluid delivery tube 14 to the patient.

A front facing cover 40 may fit over the frontward edges of the sidewalls 24 on the front side of the dividing wall 22, the cover 40 attached at its periphery to the sidewalls 24 to hold the fluid reservoir 20 within the rear volume 30 defined by the dividing wall 22, sidewalls 24, and cover 40. The cover 40 may be attached permanently or detachably to the sidewalls 24 by any number of well-known manufacturing techniques. The cover 40 may support a transparent window 45 to view the fluid reservoir 20 and whether the fluid reservoir 20 is full or empty of fluids. The sidewalls 24 or the cover 40 may include a button 46 to activate the needle injection mechanism 32 and to activate a bolus delivery as will be described.

A rear facing rear wall 42 may fit over the rearward edges of the sidewalls 24 on the rear side of the dividing wall 22, the rear wall 42 attached at its periphery to the sidewalls 24 to hold the needle injection mechanism 32 within a volume defined by the dividing wall 22, sidewalls 24, and the rear wall 42. The rear wall 42 may support an opening 58 allowing the soft needle 16 to protrude rearwardly during drug administration but remail concealed before and after drug administration.

In one embodiment, the pump housing 18 including the sidewalls 24, the dividing wall 22, and the rear wall 42 will be medical grade, rigid polymer material such as a thermoplastic fabricated by injection molding.

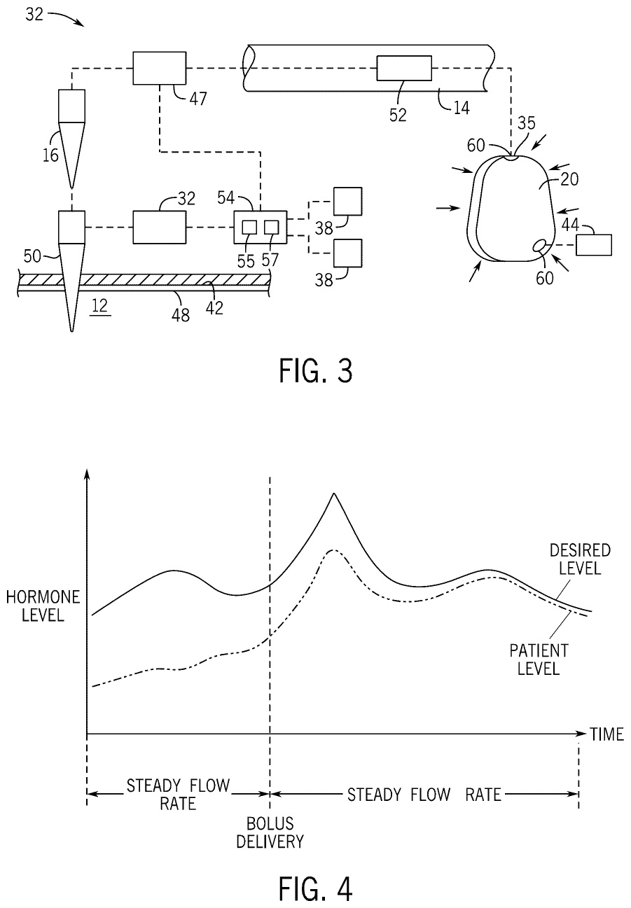

Referring to FIG. 3, the soft needle 16 may be injected under the patient's skin by the needle injection mechanism 32 supported by the rear wall 42 and the sidewalls 24 with a portion of the needle protruding rearwardly from the rear wall 42 through the opening 58 to allow for fluid hormones to flow from the fluid reservoir 20 beneath the skin during fluid hormone administration.

The fluid reservoir 20 may be filled by the pharmacist prior to or after loading the fluid reservoir 20 into the pump housing 18. A one-way flow valve 44 of conventional design permits fluid to be injected into the fluid reservoir 20 but preventing leakage backward out of the one-way flow valve 44, for example, once a drug delivery needle of a syringe has been disconnected from the one-way flow valve 44.

Within the fluid delivery tube 14, a flow restrictor 52 or capillary may regulate the flow rate of the fluid hormone using the pressure difference between the inlet and outlet. The flow rate through the flow restrictor 52 is known so that a desired dose can be delivered.

Further, a flow control valve 47 of conventional design may include flow rate control restricting or regulating the flow of fluid hormone through the control valve 47 and in this case, flow out of the fluid reservoir 20, as further discussed below. The flow control valve 47 may provide on-off regulation of flow as well as regulate the flow rate incrementally, for example, as desired by the hormone levels detected by the skin sensor 36. Thus, the fluid delivery tube 14 connected between a port 60 of the fluid reservoir 20 and the soft needle 16 is able to regulate the flow rate and dose of fluid hormone from the fluid reservoir 20 via the flow restrictor 52 and the flow control valve 47. In certain embodiments, the one-way flow valve 44 and the flow control valve 47 may be integrated into a single valve.

Prior to use by the patient 12, the fluid reservoir 20 (shown in FIG. 2) is filled by a pharmacist with reverse flow through the one-way flow valve 44 with flow of fluid hormone into the fluid reservoir 20. In one embodiment, the fluid hormone injected into the fluid reservoir 20 may be estrogen and progesterone, for example, commercially available estradiol cypionate injection or estradiol valerate forms of long-acting estrogen injection. In alternative embodiments, the fluid hormone injected into the fluid reservoir 20 may be testosterone, melatonin, insulin, growth hormone, steroid hormone, cortisol, dopamine, oxytocin, adrenaline, aldosterone, androgens, endocrine, somatostatin, prolactin, follicle stimulating hormone, thyroid stimulating hormone, adrenocorticotropic hormone, luteinizing hormone, gonadotropin-releasing hormone, lipid-derived hormones, etc. The volume of fluid hormone filled within the fluid reservoir 20 may be between 2 mL to 10 mL.

In one embodiment, as seen in FIGS. 2 and 3, the fluid reservoir 20 is an elastomeric pump with a layer of an elastic substance, natural rubber or a manmade elastic material, that is stretched by the injection of fluids into the fluid reservoir 20 to pressurize the inside of the reservoir 20. The pharmacist will clamp the port 60 of the fluid reservoir 20 or close the one-way flow valve 44 to keep the fluid reservoir 20 pressurized. When hormone delivery is desired, the clamp or valve is opened and the elastic substance will contract to its original size to push fluid out of the fluid reservoir 20.

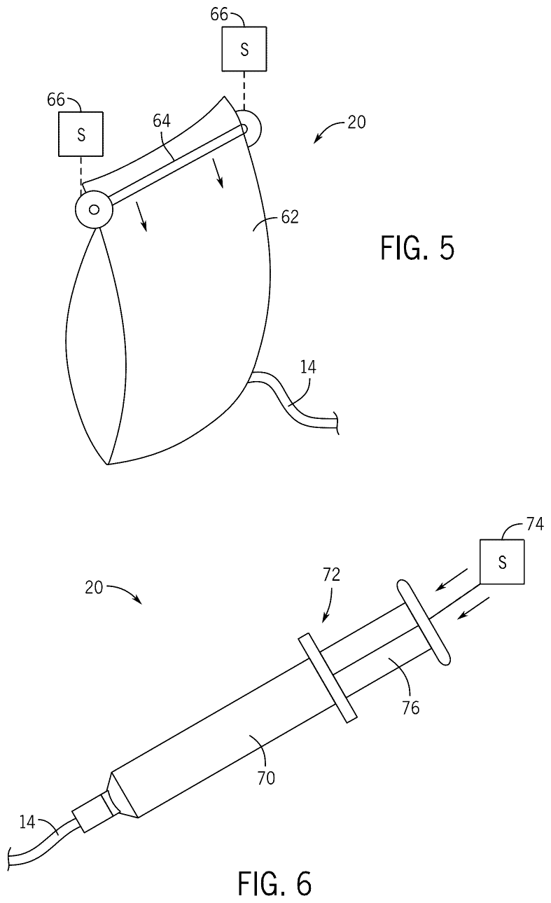

Referring briefly to FIG. 5, in another embodiment, the fluid reservoir 20 is a flexible fluid pouch 62 which is not pressurized when filled by the pharmacist. Rather, the flexible fluid bag 62 may be a medical grade plastic bag made out of polyvinyl chloride (PVC), polypropylene, ethylene vinyl acetate (EVA), copolyester ether, propyflex, and the like.

Referring briefly to FIG. 6, in another embodiment, the fluid reservoir 20 is a tubular barrel 70 of a syringe pump 72 that is filled by the pharmacist and installed with a preloaded spring 74 which is released during delivery.

Referring again to FIGS. 1 and 2, if the fluid reservoir 20 is not already loaded into the pump housing 18, the fluid reservoir 20 is loaded into the pump housing 18. The fluid delivery tube 14 is extended through the dividing wall 22 and connected to the automatic needle injection mechanism 32.

Next, the patient 12 connects the pump housing 18 to his or her skin. A double-sided adhesive sheet 48 may be attached to the rear surface of the rear wall 42 by adhesive on its first side. The adhesive sheet 48 may extend to the periphery of the sidewalls 24 and cover a central opening between the sidewalls 24 to provide a broad area of attachment to the patient's skin. The adhesive of the patient-side of the double-sided adhesive sheet 48 may be protected by a siliconized release liner 34 to be removed by the patient prior to adhering the pump housing 18 to his or her skin.

The adhesive sheet 48 will desirably be breathable and oxygen permeable to allow for long-term attachment to the patient 12 while preserving skin health and for this purpose, the pump housing 18 allows for the infiltration of outside air to improve oxygen transmission through the adhesive sheet 48, this outside air passing through vents within the sidewalls 24 of the pump housing 18 and/or through vent openings in the dividing wall 22.

The adhesive sheet 48 may include a skin sensor 36. The skin sensor 36 may communicate with control electronics 54 to only allow the automatic needle injection mechanism 32 to deploy if the pump housing 18 is attached to the skin. The adhesive sheet 48 may further include other body sensors 38 such as a temperature sensor, heart rate sensor, respiratory rate sensor, and blood oxygen sensor used to detect bioindicators of hormone level such as skin temperature, blood pressure, galvanic skin response, heart rate, respiration variability, and the presence of certain biomarkers in the patient's sweat or blood. The body sensors 38 may be integrated with the adhesive sheet 48, supported on the pump housing 18 or separate from the pump housing 18 as shown to communicate wirelessly, e.g., using Bluetooth, with control electronics 54 permitting real time control of the drug administration dose and flow rate as further described below.

Referring again to FIG. 3, once the wearable injector pump 10 is adhered to the skin of the patient 12, the automatic needle injection mechanism 32 in activated by the patient 12 to, for example, inject a stiff stylet 50 such as a standard stainless steel hypodermic needle for intravenous drug administration into the patient's skin using a mechanical injection mechanism 32 known in the art, such as preloaded springs, and allowing the coaxially extending soft needle 16, typically a relatively flexible polymer cannula, to pierce the skin simultaneously with the stiff stylet 50.

A number of different mechanisms may be used to implement the needle injection mechanism to insert the stiff stylet 50 and soft needle 16 simultaneously and then retract and remove the stiff stylet 50 rapidly. In one embodiment, the stiff stylet 50 and soft needle 16 are inserted by manually pressing the button 46 on the front cover 40 or sidewalls 24 that releases a helical preloaded compression spring which accelerates the stiff stylet 50 and soft needle 16 rearward puncturing the skin. Other mechanisms including motor driven mechanisms and manually operated injection systems requiring manual injections by the patient are also contemplated.

After the needle piercing operation, the stiff stylet 50 is automatically or manually retracted, for example, using a similar preloaded spring mechanism leaving only the soft needle 16 in place as will be discussed in more detail below. By removing the stiff stylet 50 in favor of the soft needle 16, improved patient comfort may be obtained during insertion of the soft needle 16. In one embodiment, the stiff stylet 50 is eliminated and the soft needle 16 is a rigid needle that softens and becomes flexible after it enters the body.

The fluid hormone may be pumped out of the fluid reservoir 20 using several different pumping mechanisms including elastomeric bladders, constant force springs, syringe pumps, piezoelectric pumps, peristaltic pumps, and the like.

In one embodiment, as seen in FIGS. 2 and 3, the fluid reservoir 20 is an elastomeric bladder which may move the fluid hormone from the fluid reservoir 20 through the fluid delivery tube 14 to the soft needle 16 by releasing the clamp and/or opening the flow control valve 47 allowing for the continuous contraction of the elastomeric bladder.

In another embodiment, as seen in FIG. 5, the fluid reservoir 20 is a flexible fluid pouch 62 that interacts with a roller 64 that moves with pretensioned springs 66, e.g., constant force springs, that apply a force on the roller 64 to roll along the flexible fluid pouch 62 to push fluid out of the fluid reservoir 20 through the fluid delivery tube 14 to the soft needle 16. The flow control valve 47 can change the flow rate through the fluid delivery tube 14 and the soft needle 16.

In yet another embodiment, as seen in FIG. 6, the fluid reservoir 20 is a tubular barrel 70 of a syringe 72 that is attached to a plunger 76 with an elastomeric piston at the end of the plunger 76 moveable to displace the fluid within the tubular barrel 70 and discharge the fluid within the tubular barrel 70 through the fluid delivery tube 14 to the soft needle 16. The plunger 76 may extend outward from the pump housing 18 or be held within the pump housing 18 and force may be applied to the plunger 76 using a pretensioned spring 74 applying a pressure on the plunger 76 to expel fluid through the fluid delivery tube 14 to the soft needle 16. Again, the flow control valve 47 can change the flow rate through the fluid delivery tube 14 and the soft needle 16.

Referring to FIG. 4, the flow restrictor 52 may restrict the flow rate of fluid from the fluid reservoir 20 to a predetermined flow rate while the flow control valve 47 may regulate the rate and dose amount of fluid hormone released from the fluid reservoir 20. The amount of fluid hormone pumped from the fluid reservoir 20 may be based on desired hormone levels and created desired set points or targets for hormone levels as further described below.

The desired hormone levels may be based on a historical average of anonymized patients (based on age, height and weight of the patient) or historical average of the specific patient. The desired hormone levels may track expected hormone fluctuations in the patient 12, for example, during the patient's menstrual cycle so that the desired hormone levels mimic expected biological cycles. Representative data of anonymized patients with similar age, height and weight may be used when historical data of the specific patient is unavailable. The desired hormone levels may be data stored in electronic memory 55 and executed by a program 57 stored in memory of the control electronics 54.

The hormone levels of the actual patient 12 may be sensed in real time by a closed loop feedback system. The skin sensor 36 and/or body sensors 38 attached to the pump housing 18 or external to the wearable injector pump 10 may communicate with the control electronics 54 to measure the real time hormone levels, compare the real time hormone levels and desired hormone levels, and adjust the flow rate of hormones from the fluid reservoir 20 by regulating the flow control valve 47. When real time sensing is unavailable, the expected hormone deficits in the actual patient 12 may be based on prior clinical data and testing on the patient 12.

The control electronics 54 may be programmed to monitor and control the opening and closing of the control valve 47, for example, to control the rate and dose of fluid hormones delivered to the patient 12 and the timing of the dose delivery. In one embodiment, an open loop control system is used and the control electronics 54 will adjust the opening and closing of the control valve 47 based on desired set points or targets for hormones and schedules of average or known hormone levels (hormone deficits) of the patient 12. In an alternative embodiment, a closed loop control system is used and the control electronics 54 will communicate with various body sensors, for example, the skin sensor 36 and body sensors 38, to monitor the hormone levels of the actual patient 12 in real time. The actual hormones levels may be estimated based on the patient's biomarkers. Thus, the control electronics 54 will adjust the control valve 47 based on a desired set points or targets for the hormones and on the patient's estimated or actual hormone levels.

The control electronics 54 will also detect large drops in hormone levels (or a big spike in hormone levels) in which a bolus or single large dose of fluid hormone will be delivered to the patient. The bolus may also be requested by the patient 12 by pressing a bolus button 56 communicating with the control electronics 54, for example, when the patient 12 is experiencing symptoms and needs immediate physiological response. Otherwise, smaller amounts of fluid hormone will be delivered over time at more steady flow rates to reduce the side effects of large fluctuations in hormone level when boluses are delivered. The control electronics 54 will detect decreasing trends in hormones levels in the patient in which smaller amounts of fluid hormone can start to be delivered to anticipate decreases in hormone level. The control electronics 54 will detect increasing trends in hormone levels to stop the delivery fluid hormones to prevent overshooting the desired set points or target levels.

The control electronics 54 may use predictive artificial intelligence (AI) technologies to predict the fluctuations of the patient's hormone levels in a manner which anticipates drops in hormone level before they occur and therefore deliver fluid hormone before the physical side effects are felt by the patient. For example, AI may be used to determine what the desired levels of hormones are in the body and to supplement the hormones levels of the actual patient 12 to reach the desired levels in a manner which mimics a healthy human patient. In this respect, AI provides enhanced control of the control electronics 54 by reacting in a manner which predicts hormone trends in the patient 12, adapts to unexpected hormone changes, and compensates for low hormone levels by mimicking the hormone levels and natural cycles of the healthy human patient. It is understood that hormones levels naturally fluctuate in cycles, for example, menstrual cycle, and therefore hormone cycles and natural fluctuations can be detected by AI and distinguished from other hormone level changes. AI can detect small biological changes or detect imperceptible trends and react much more quickly than traditional methods.

Following drug administration, the soft needle 16 may be manually or automatically retracted into the pump housing 18. The soft needle 16 may be retracted using a preloaded spring mechanism known in the art. In one embodiment, a sensor such as a capacitive sensor or ultrasonic sensor may detect that the fluid reservoir 20 is empty and automatically retract the soft needle 16 indicating the completion of drug administration. The stiff stylet 50 and soft needle 16 are concealed before and after drug administration to eliminate accidental needle sticks.

The empty fluid reservoir 20 may be removed from the skin of the patient 12 by removing the adhesive sheet 48 and pump housing 18 from the skin of the patient 12. The pump housing 18 may be disposed of as a single use device or the empty fluid reservoir 20 may be removed from the pump housing 18 by the pharmacist and the pump housing 18 may be reused by the patient with a new fluid reservoir 20 filled by the pharmacist.

It is contemplated that the injector pump may be used to deliver liquid drugs which not only include hormones but may include other drugs mixed with hormones or drugs without hormones.

It is contemplated that the injector pump may be a wearable pump or an ambulatory infusion pump known in the art which permits the delivery of drugs under a controlled rate.

Certain terminology is used herein for purposes of reference only, and thus is not intended to be limiting. For example, terms such as “upper”, “lower”, “above”, and “below” refer to directions in the drawings to which reference is made. Terms such as “rear”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What I claim is:

1. A method of delivering hormones to a human patient, the method comprising:
attaching an ambulatory pump to a skin of the human patient, the pump having a pump housing attachable to the skin of the human patient, a hypothermic needle supported by the pump housing along an insertion path of the hypothermic needle into the skin of the human patient, a fluid container supported by the pump housing and containing liquid hormones to fluidly communicate with the hypothermic needle to deliver liquid hormones to the hypothermic needle, a flow control valve controlling the flow of liquid hormones through the hypothermic needle, and a controller sending a signal to the flow control valve to control an amount of flow of the liquid hormones through the flow control valve;
monitoring real time hormone levels of the human patient; and
delivering liquid hormones and adjusting the flow of liquid hormones based on comparing real time hormone levels of the human patient and desired set points or targets for hormones levels within the human patient.

2. The method of claim 1 further comprising detecting changes in the hormone level and distinguishing from predictable fluctuations expected in the human body.

3. The method of claim 2 further comprising detecting decreases in hormones level and increasing the flow of liquid hormones to the human patient.

4. The method of claim 3 wherein increasing the flow of liquid hormones to the human patient is at a steady rate over an extended period.

5. The method of claim 4 wherein increasing the flow of liquid hormones to the human patient further includes a single bolus to the human patient.

6. The method of claim 5 wherein delivering the bolus to the human patient is in response to a request from the human patient by pressing a button.

7. The method of claim 1 further comprising using predictive artificial intelligence (AI) technologies to predict changes in a patient's hormone levels before they occur.

8. The method of claim 1 wherein the ambulatory pump further comprises an electronic memory storing the desired set points or targets for hormones levels within the human patient.

9. The method of claim 8 wherein the desired set points or targets for hormone levels of the patient is based on a historical average of the human patient.

10. The method of claim 9 wherein the historical average of the human patient is based on prior clinical data and testing on the specific human patient.

11. The method of claim 8 wherein the desired set points or targets for hormone levels of the patient is based on a historical average of anonymized patient data based on similar based on age, height and/or weight.

12. The method of claim 1 further comprising monitoring the hormone level of the human patient using a body sensor.

13. The method of claim 12 wherein the body sensor is a skin sensor measuring body markers of the human patient.

14. The method of claim 13 wherein the body sensor is attached to the pump housing.

15. The method of claim 13 wherein the body sensor is external to the pump housing.

16. An ambulatory pump attachable to skin of a human patient comprising:
a pump housing attachable to the skin of the human patient;
a hypothermic needle supported by the pump housing along an insertion path of the hypothermic needle into the skin of the human patient;
a fluid container supported by the pump housing and containing liquid hormones to fluidly communicate with the hypothermic needle to deliver liquid hormones to the hypothermic needle;
a flow control valve controlling the flow of liquid hormones through the hypothermic needle; and
a controller sending a signal to the flow control valve to control an amount of flow of the liquid hormones through the flow control valve based on desired set points or targets for hormones levels within the human patient.

17. The ambulatory pump of claim 16 further comprising an electronic memory storing the desired set points or targets for hormones levels within the human patient.

18. The ambulatory pump of claim 17 wherein the desired set points or targets for hormones levels within the human patient consider predictable fluctuations expected in the human body.

19. The ambulatory pump of claim 18 wherein the fluid container is a flexible fluid pouch.

20. The ambulatory pump of claim 18 wherein the fluid container is a syringe.
